(19) United States
(12) Patent Application Publication
(10) Pub. No.: US 2014/0366251 A1
(43) Pub. Date: Dec. 18, 2014

(54) EAR GUARD

(71) Applicant: JoAnn Anderson, Brooklyn, NY (US)

(72) Inventor: JoAnn Anderson, Brooklyn, NY (US)

(21) Appl. No.: 14/307,464

(22) Filed: Jun. 17, 2014

Related U.S. Application Data
(60) Provisional application No. 61/835,769, filed on Jun. 17, 2013.

(51) Int. Cl. A61F 11/14; A61F 11/06; A42B 3/00

(52) U.S. Cl. CPC A61F 11/14; A42B 3/00; A61F 11/06
USPC 2/410

(57) ABSTRACT

The invention provides an ear guard that protects both the inner and outer ear from exposure to chemicals and excess water when caring for the hair. The ear guard is fabricated of a durable yet pliable rubber or plastic material. Essentially oval and ear shaped, the ear guard is made to accommodate the entire ear, like a glove on the hand, comprising an elastic band around its perimeter to ensure a secure fit while in use.

### CLAIM OF PRIORITY

This patent application claims priority under 35 USC 119 (e) (1) from U.S. Provisional Patent Application Ser. No. 61/835,769 filed Jun. 17, 2013, of common inventorship herewith entitled, “Ear Guard.”

### BACKGROUND OF THE INVENTION

1. Field of the Invention

This invention relates generally to an ear guard and, more particularly, the invention relates to an ear guard protects both the inner and outer ear from exposure to chemicals and excess water when caring for the hair.

2. Description of the Prior Art

Thousands of men and women across the country enjoy a fun and rewarding career as a professional barber or hairstylist. Styling hair can be an exciting way to meet new, interesting people and earn great money in the process. For most conscientious stylists, a foremost concern when dealing with clients is to carefully execute a style, cut, or color in a manner that is both comfortable and safe for the client. Unfortunately, the some of the implements utilized in these endeavors, such stringent chemicals, can be quite hazardous; thus, stylists must be extra careful in order to ensure the well-being of their customer. One area in particular that is at risk for injury when having the hair styled are the ears. Sticking out from the sides of the head, the ears are sometimes an unavoidable obstacle for stylists and many caring professionals have accidentally drizzled colorings and permanent solutions onto and into the ears of their clients. As can be imagined, potentially harming a client when attempting to execute a fashionable style can be both frustrating for the client and unsettling for the stylist. Even a simple shampooing can fraught with challenges, as the hair product, not to mention submersion of the head in water, can lead to painful invasion of the ear canal. While these are common obstacles in the salon environment, those who style their own hair at home often face the same challenges when it comes to protecting their ears.

### BRIEF DESCRIPTION OF THE DRAWINGS

FIG. 1 is perspective view showing how the ear of the present invention fits over a human ear.

FIG. 2 is a perspective view showing the ear guard of the present invention in place covering a human ear.

### DETAILED DESCRIPTION OF THE PREFERRED EMBODIMENTS

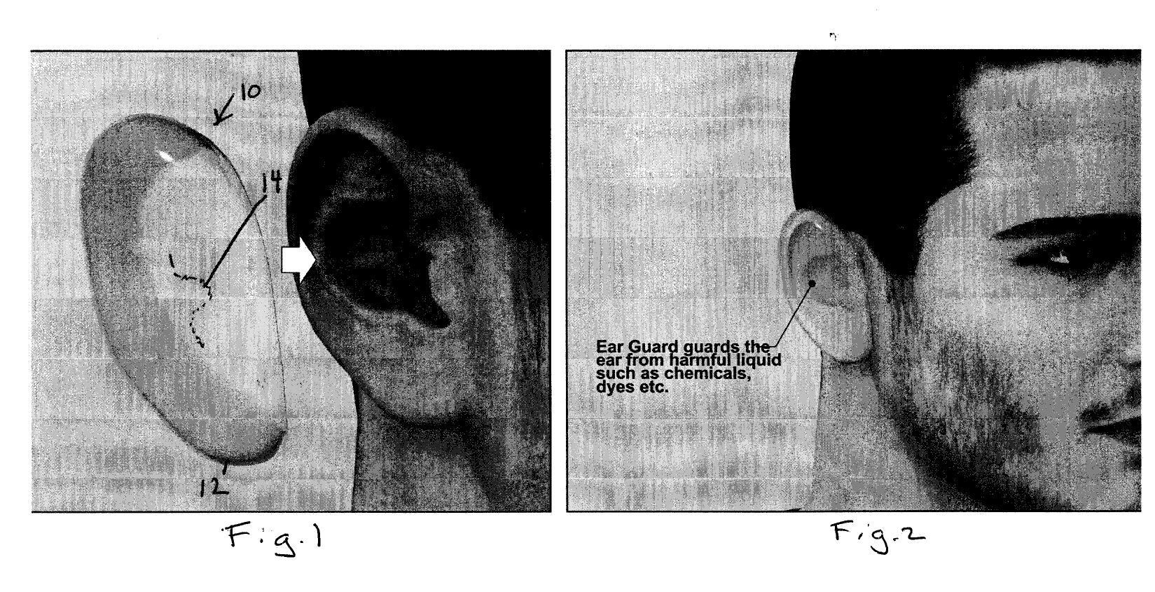

As illustrated in FIGS. 1 and 2, the present invention is an ear guard that protect both the inner and outer ear from exposure to chemicals and excess water when caring for the hair.

The ear guard 10 of the present invention is fabricated of a durable yet pliable rubber or plastic material. Essentially oval and ear shaped and made to accommodate the entire ear, like a glove on the hand, as shown in FIG. 1, each ear guard contains an elastic band 12 around its perimeter to ensure a secure fit while in use.

For optimal inner ear protection, in an embodiment, each ear guard also features an internal plug device 14 that comfortably fits inside the ear. The guard is offered in a variety of sizes to accommodate all ear dimensions and types. Conveniently disposable, the ear guard can be sold in bulk with up to fifty (50) units per package. As an additional consideration, the guard can also be made available as reusable units.

Very simple in design, use of the ear guard of the present invention is simple and straightforward. Before undergoing any hair treatment, a user stretches the elastic band of the cap so that it fully encompasses the ear from the top to the bottom of the earlobe. The internal plug 14 is fitted inside the ear. This process is repeated for the other ear. While in use, the ear guard deflects away from the ears any liquids that are applied to the head, from water and shampoo to colorings and bleaches. Once the hair is dry, a user removes each cap from the ears and discards it in the closest garbage receptacle.

The ear guard of the present invention offers professional stylists and their clientele a number of significant benefits and advantages. Foremost, the guard provides stylists a simple and efficient means of protecting their client's ears from the many hazardous liquids utilized to style the hair. An easily applied protective sheath, the durable guard serves as an impenetrable barrier between the fragile and sensitive ear and the solutions utilized to color or bleach the hair. Encompassing the ears completely, inside as well as out, the guard also deflects running water, as well as dripping or running shampoos, and any other processing chemicals on contact, thus preventing these agents from clogging the inner ear and causing discomfort to the customer. Sparing the client the hassle and pain that can result from accidents with long-lasting effects, use of the guard ensures that the user can have their washed, styled, and otherwise treated in a comfortable and safe manner. Allowing the salon professional to provide supreme service to their clients, use of the guard can result in greater customer satisfaction and repeat business. Perfect for salons, the guard can also be utilized in the home as a means of protecting the ears when washing the hair and applying home-based perms. Highly versatile, the guard can be worn while taking a shower, ensuring that the relentless flow of water over the head does not enter the ears. The guard can also be employed by those who spend extended periods outdoors, as it could protect the ears from exposure to harmful ultraviolet rays from the sun.

Practical and unique, the ear guard of the present invention offers professional stylists and general users alike a simple and effective means of protecting the ears from hazardous styling products as well as water damage. Simple to use and durably constructed, the guard proves a valuable commodity to any professional stylist or colorist and is also ideal for general use.

The foregoing exemplary descriptions and the illustrative preferred embodiments of the present invention have been explained in the drawings and described in detail, with varying modifications and alternative embodiments being taught. While the invention has been so shown, described and illustrated, it should be understood by those skilled in the art that equivalent changes in form and detail may be made therein without departing from the true spirit and scope of the invention, and that the scope of the present invention is to be limited only to the claims except as precluded by the prior art. Moreover, the invention as disclosed herein may be suitably practiced in the absence of the specific elements which are disclosed herein.

1. An ear guard device to protect an ear from chemicals and dyes during hair treatment comprising: an essentially oval ear shaped ear guard fabricated of a durable yet pliable rubber or plastic material.

2. The ear guard device of claim 1, further comprising an elastic band around the perimeter of the ear guard to ensure a secure fit while in use.

3. The ear guard device of claim 1, further comprising an internal plug device that comfortably fits inside the ear.

4. The ear guard device of claim 2, further comprising an internal plug device that comfortably fits inside the ear.

5. The ear guard device of claim 1, wherein the ear guard is made in a variety of sizes to accommodate all ear dimensions and types.

6. The ear guard device of claim 2, wherein the ear guard is made in a variety of sizes to accommodate all ear dimensions and types.

7. The ear guard device of claim 3, wherein the ear guard is made in a variety of sizes to accommodate all ear dimensions and types.

8. The ear guard device of claim 4, wherein the ear guard is made in a variety of sizes to accommodate all ear dimensions and types.
